(19) United States
(12) Patent Application Publication
(10) Pub. No.: US 2014/0293586 A1
(43) Pub. Date: Oct. 2, 2014

(54) Detachable Essential Oil Ring for Lamp and Application Thereof

(71) Applicant: Shuoen Tech. Co., Ltd., Miaoli City (TW)

(72) Inventor: Chung-Chi Chiang, Miaoli City (TW)

(73) Assignee: Shuoen Tech. Co., Ltd., Miaoli City (TW)

(21) Appl. No.: 14/226,873

(22) Filed: Mar. 27, 2014

(30) Foreign Application Priority Data
Mar. 28, 2013 (TW) 102205736

(51) Int. Cl. A61L 9/03

(52) U.S. Cl. CPC A61L 9/03
USPC 362/101; 422/125

(57) ABSTRACT

A detachable essential oil ring for lamp is an open-loop ring and is detachably mounted on the lamp easily. A part of the detachable essential oil ring is used to absorb or contain essential oils and directly contacts a heated portion of the lamp. The detachable essential oil ring contacts the heated portion to auxiliary vaporize the fragrance oils or the essential oils. The structure of the detachable essential oil ring is simple and is easy to manufacture. The detachable essential oil ring can be mounted on the lamp with different types and can auxiliary vaporizes the fragrance oils or the essential oils by the heat generated from the lamp during light, and this is lower cost and environmental friendly.

### BACKGROUND OF THE INVENTION

1. Field of the Invention

The present invention relates to a ring for coating or storing fragrance oils or essential oils, and more particularly to a detachable essential oil ring that can be detachably mounted on a lamp and can auxiliary vaporize the fragrance oils or the essential oils by the heat generated during lighting.

2. Description of Related Art

Many studies have shown that special smell and fragrance can greatly help users to enhance the quality of work, the mental health and the stability of mind. Therefore, many products that are used to auxiliary vaporize fragrance oils or essential oils have appeared on the market, such as flavors wax, ultrasound fragrance spray device, combustion flavors light, thermal-photoelectric-heating flavor release device and so on. However, the conventional flavor release devices are usually belonging to energy-consumption-type products and are needed to increase additional energy such as electric heating, thermal radiation (incandescent light) or combustion to assist the release or the volatilization of fragrance oils.

Although the conventional flavor release devices can provide a releasing effect to the surroundings, almost all of the conventional flavor release devices required extra energy, and this is not environmental friendly. The costs of using the conventional flavor release devices are expensive and they are inconveniently in use.

To overcome the shortcomings, the present invention provides a detachable essential oil ring for lamp and a lamp with detachable essential oil ring to mitigate or obviate the aforementioned problems.

### SUMMARY OF THE INVENTION

The main objective of the present invention is to provide a ring for coating or storing or absorbing fragrance oils or essential oils, and more particularly to a detachable essential oil ring that can be detachably mounted on a lamp and can auxiliary vaporizes the fragrance oils or the essential oils by the heat generated from the lamp.

The present invention is related to a detachable essential oil ring for lamp and a lamp with detachable essential oil ring. The detachable essential oil ring is an open-loop ring and is detachably mounted on the lamp easily. A part of the detachable essential oil ring is used to absorb or contain essential oils. The detachable essential oil ring containing essential oil is attached to a heated portion of the lamp and then auxiliary vaporize the fragrance oils or the essential oils. The structure of the detachable essential oil ring is simple and is easy to manufacture. The detachable essential oil ring can be mounted on the lamp with different types and can auxiliary vaporizes the fragrance oils or the essential oils by the heat generated from the lamp during lighting, and this is lower cost and environmental friendly.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a lamp with a first embodiment of a detachable essential oil ring in accordance with the present invention;

FIG. 2 is an exploded perspective view of the lamp in FIG. 1;

FIG. 3 is an enlarged side view of the detachable essential oil ring in FIG. 1;

FIG. 4 is an enlarged bottom perspective view of the detachable essential oil ring in FIG. 1;

FIG. 5 is an enlarged bottom view of the detachable essential oil ring in FIG. 1;

FIG. 6 is an enlarged perspective view of a second embodiment of a detachable essential oil ring in accordance with the present invention;

FIG. 7 is a bottom view of the detachable essential oil ring in FIG. 6;

FIG. 8 is an enlarged perspective view of a third embodiment of a detachable essential oil ring in accordance with the present invention;

FIG. 9 is a bottom view of the detachable essential oil ring in FIG. 8;

FIG. 10 is an enlarged perspective view of a fourth embodiment of a detachable essential oil ring in accordance with the present invention; and

FIG. 11 is a side view of the detachable essential oil ring in FIG. 10.

### DETAILED DESCRIPTION OF THE PREFERRED EMBODIMENT

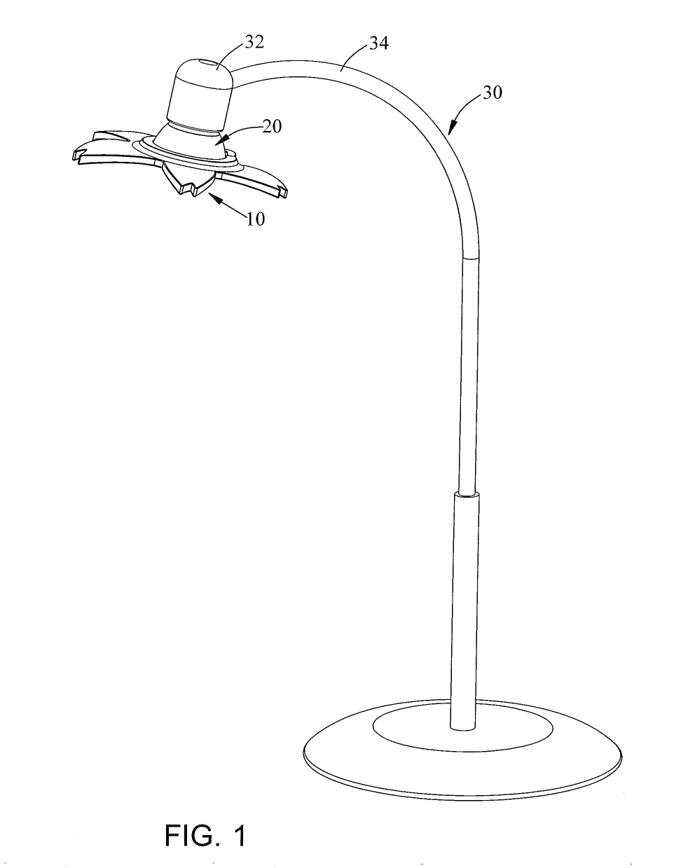

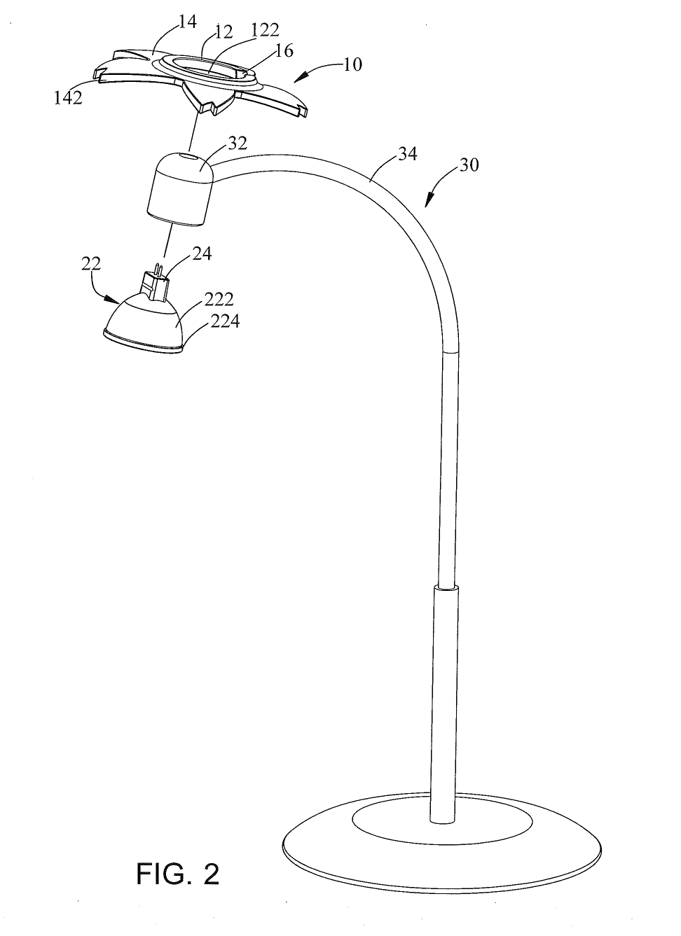

With reference to FIGS. 1 and 2, a detachable essential oil ring for lamp in accordance with the present invention, the lamp comprises a connecting base 30, a source device 20 and a detachable essential oil ring 10.

The connecting base 30 has a top, a supporting arm 34 and a lamp-holder 32. The supporting arm 34 is mounted on and extends upwardly from the top of the connecting base 30 and has an upper end. The lamp-holder 32 is securely mounted on the upper end of the supporting arm 34 and has a bottom flange.

The source device 20 is mounted on and electrically connected to the lamp-holder 32, and emits light by an external electric power transmitting via the lamp-holder 32. The source device 20 has a connector 24, a radiating shade 22, a heated portion and a light source (not shown in the drawings).

The connector 24 is mounted in and electrically connected to the lamp-holder 32 and has a bottom.

The radiating shade 22 is connected to the connector 24, abuts the lamp-holder 32 and has a top, a bottom, an external surface, a heat-dissipating face 222 and an extending flange 224. The shape and the material of the radiating shade 22 are not limited. A location of the heated portion may be corresponding to the radiating shade 22 and the heated portion has relatively higher temperature during lighting. The top of the radiating shade 22 is connected to the bottom of the connector 24. The heat-dissipating face 222 may be semicircular, is formed on the external surface of the radiating shade 22 and abuts the bottom flange of the lamp-holder 32. The extending flange 224 is formed on and protrudes from the heat-dissipating face 222 at the bottom of the radiating shade 22. Preferably, the radiating shade 22 may be made material with high thermal conductivity material, such as thermal-conductive polymer or metal. Additionally, the thermal-conductive polymer may be a modified polymer composite material with carbon material or thermal-conductive material therein.

The light source may be an incandescent light or a light emitting diode (LED), is mounted in the radiating shade 22 and is electrically connected to the connector 24. The light source can emit light powered by an external electric power source via the lamp-holder 32 and the connector 24. When the light source emits light while generating thermal energy, an arrangement between the radiating shade 22 and the powered light source generates the thermal energy to the radiating shade 22 by thermal conductivity or thermal radiation to cooling the lamp.

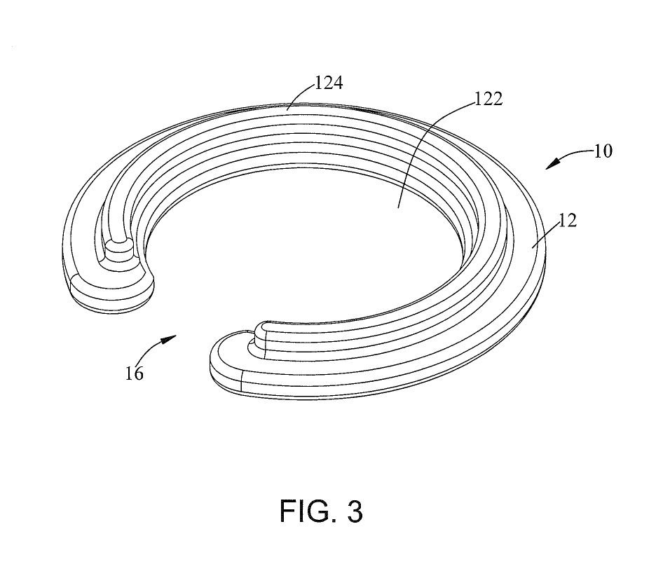

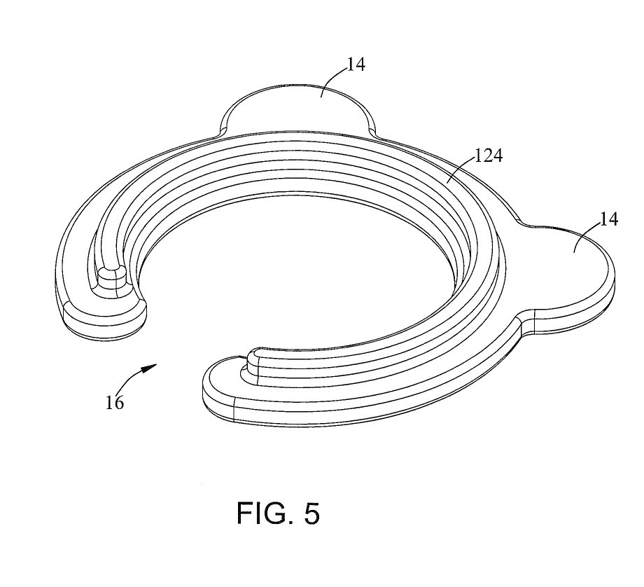

With reference to FIGS. 3, 4 and 5, a first embodiment of the detachable essential oil ring 10 is detachably mounted on the heated portion of the source device 20 and has a body 12 and a notch 16. The body 12 may be a C-shaped ring that has a thermal conductivity performance, absorb essential oils or made of a material containing essential oils. In addition, the body 12 may be made of pottery, clay, ceramic materials, gypsum, porous thermally conductive materials or carbon materials that absorbs or contains the essential oils.

The body 12 is movably mounted on the heated portion of the heat-dissipating face 222 of the radiating shade 22 and has a top side, a bottom side, a center and a mounting recess 122. Preferably, a part of the top side of the body 12 is made of ceramic materials without glazes, so as to allow a part of the top side of the body 12 to absorb the fragrance oils or the essential oils. The mounting recess 122 is formed through the top side and the bottom side of the body 12 at the center of the body 12 to enable the body 12 to mount on the radiating shade 22. The notch 16 is formed through the top side and the bottom side of the body 12 and communicates with the mounting recess 122 to form the C-shaped body 12. By the mounting recess 122 and the notch 16, the detachable essential oil ring 10 can be easily mounted through the supporting arm 34 and the lamp-holder 32 to mount on the radiating shade 22. Then, the body 12 of the detachable essential oil ring 10 abuts the heat-dissipating face 222 of the radiating shade 22 to enable the thermal energy that generates from the light source to conduct to the body 12. The fragrance oils or the essential oils being absorbed or being contained in the body 12 can be volatilized into the air by the auxiliary of residual heat to produce aroma.

Preferably, glazes or any auxiliary thermal materials can be coated on the body 12 and the heat-dissipating face 222 of the radiating shade 22 so as to enable the residual heat being quickly transferred into the body 12.

Furthermore, in order to enhance the content of the essential oils in the body 12, or enhance the distributing efficiency of the essential oils, or improve the appearance of the lamp or improve the connection between the body 12 and the radiating shade 22, the detachable essential oil ring 10 further has a protruding segment 14. The protruding segment 14 is formed on and protrudes from a periphery of the body 12 and has multiple sheet elements. Preferably, the sheet elements are radially formed on and protrude from the periphery of the body 12. In addition, each one of the sheet elements has a various shape and may be polygonal, semicircular or curved, and this can enable the detachable essential oil ring 10 to present with different appearances such as expanded petals, unfolding leaves, budding flowers, flags or cute animals, etc. In the first embodiment, each one of the sheet elements of the protruding segment 14 is cherry petal-shaped as shown in FIGS. 3 to 5.

Furthermore, the protruding segment 14 can be connected to the body 12 by engaging, sticking or tightening and can be made of ceramic materials. In addition, glazes, paints, any protection layers or isolation layers can be coated or mounted on the protruding segment 14 to protect the cleanliness of the protruding segment 14 without polluting by the essential oils.

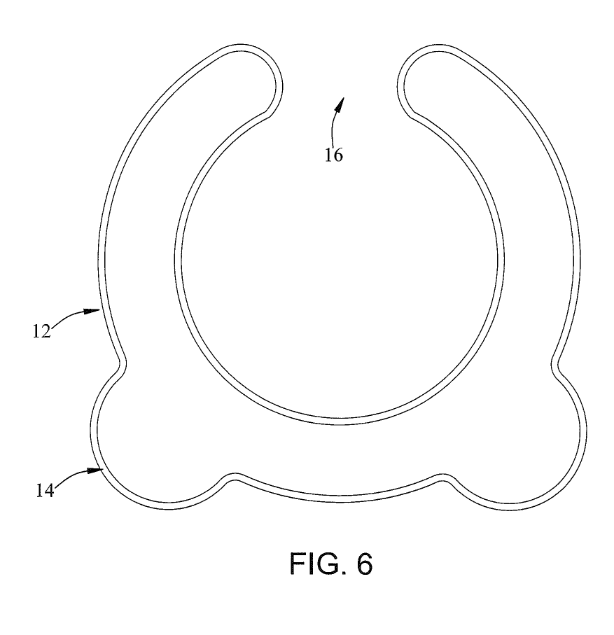

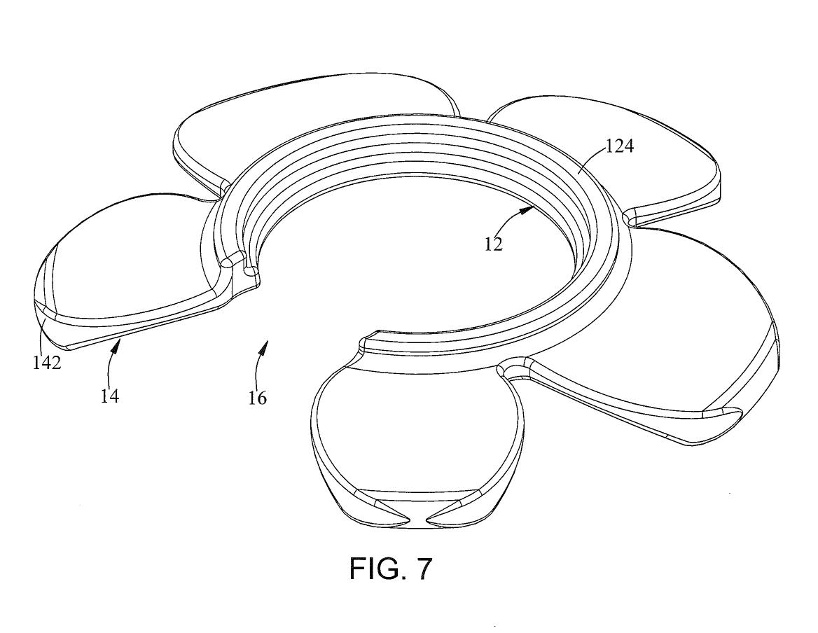

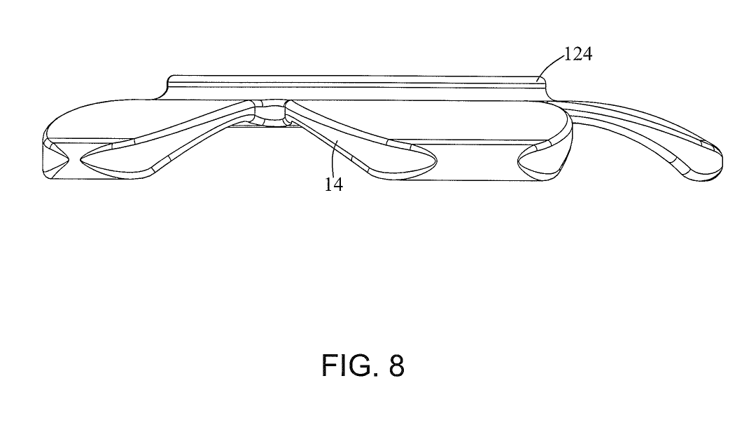

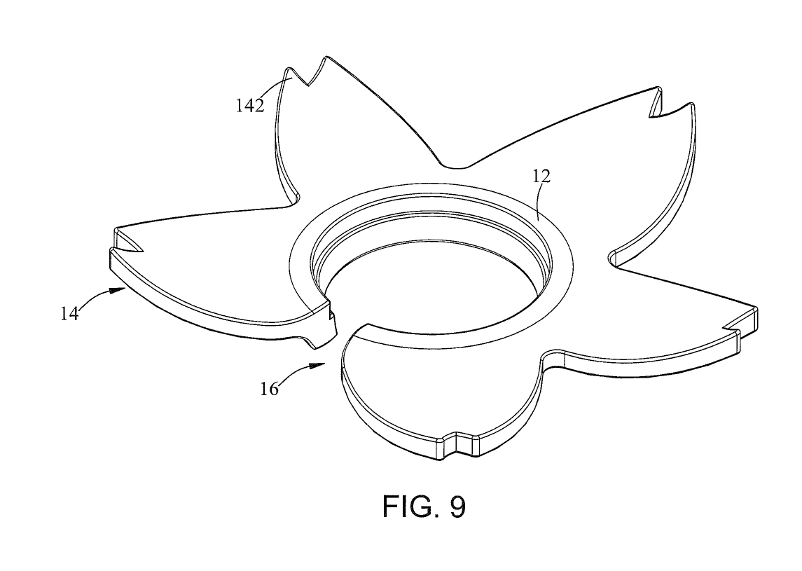

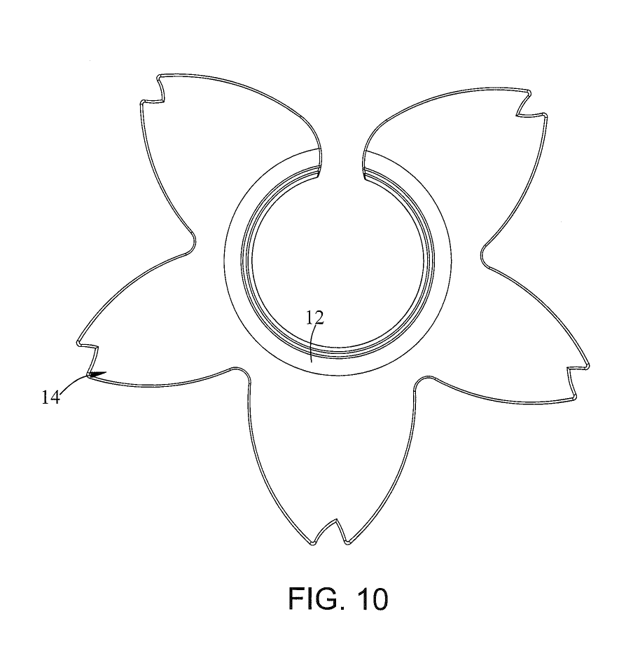

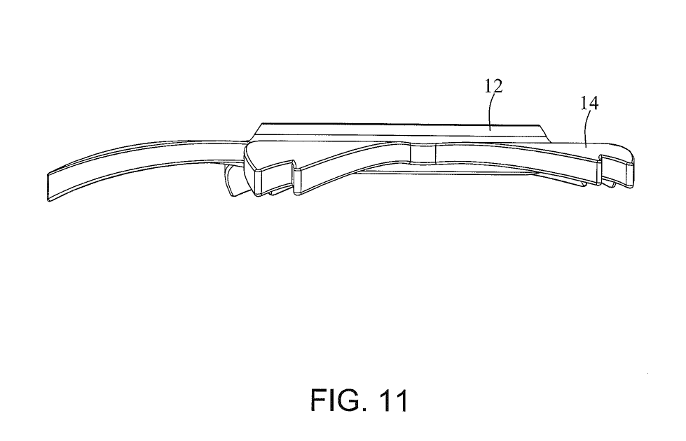

With reference to FIGS. 6 and 7, a second embodiment of the detachable essential oil ring 10 is substantially the same as the first embodiment except the following features. The second embodiment of the detachable essential oil ring 10 does not have the protruding segment 14. With reference to FIGS. 8 and 9, a third embodiment of the detachable essential oil ring 10 is substantially the same as the first embodiment except the following features. Each one of the sheet elements of the protruding segment 14 of the third embodiment of the detachable essential oil ring 10 is circular. With reference to FIGS. 10 and 11, a fourth embodiment of the detachable essential oil ring 10 is substantially the same as the first embodiment except the following features. Each one of the sheet elements of the protruding segment 14 of the third embodiment of the detachable essential oil ring 10 is petal-shaped.

Additionally, the body 12 of the second embodiment, the third embodiment and the fourth embodiment of the detachable essential oil ring 10 further has an abutting segment 124 axially formed on and protruding from the top side of the body 12 to enable the body 12 and the abutting segment 124 to closely abut the heat-dissipating face 222 of the radiating shade 22 when the detachable essential oil ring 10 is mounted on the source device 20, and this. provide a preferred conduction of the detachable essential oil ring 10. Furthermore, the abutting face 124 has a side face abutting the radiating shade 22 with glazes or thermal conductive layers.

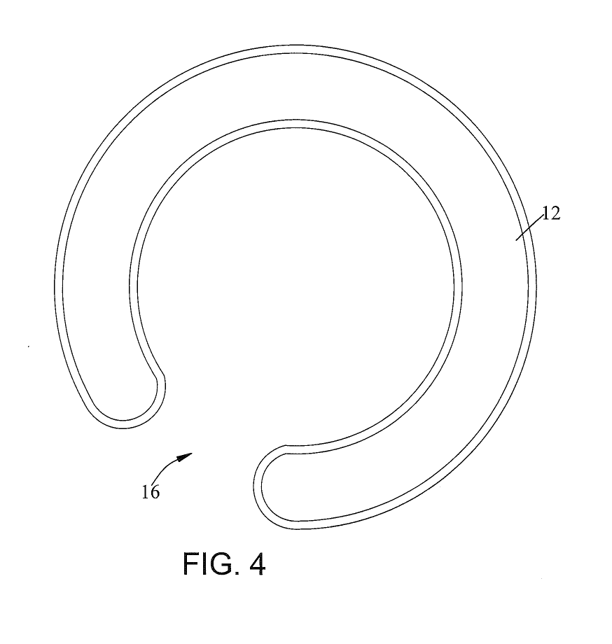

In the first embodiment and the fourth embodiment of detachable essential oil ring 10, each one of the sheet elements of the protruding segment 14 has a counterweight end 142. The structure of the counterweight end 142 of each one of the sheet elements is not limited, and may be increased the thickness of the counterweight end 142 as shown in FIGS. 10 and 11 or may be increased the length of the counterweight end 142 or may be bent the counterweight end 142 as shown in FIGS. 3 and 4. The counterweight ends 142 of the sheet elements of the protruding segment 14 may enhance the detachable essential oil ring 10 to securely and stably mount on the source device 20 due to unbalanced weight or displacement.

In use, the detachable essential oil ring 10 is connected with the source device 20 and the connecting base 30 to enable the lamp to have at least two functions of lighting effect and aroma-releasing effect. Additionally, the detachable essential oil ring 10 can be easily mounted on the source device 20 and can be connected with other components of the lamp visually.

According to the above-mentioned description, a detachable essential oil ring 10 for lamp and a lamp with detachable essential oil ring 10 as described have the following advantages.

1. The structure of the detachable essential oil ring 10 is simple and is easy to manufacture. The detachable essential oil ring 10 can be easily mounted on a lamp of different kinds or types. The fragrance oils or the essential oils that absorb or contain on the detachable essential oil ring 10 can be vaporized into the air by residual heat that generates from the lamp, and this can completely solve the problems of inconvenient using, consuming extra energy and high prices of the conventional flavor release devices.

2. The detachable essential oil ring 10 can be made with various shapes according to the user's requirements and can be integrated into the source device 20 to provide an aesthetic outcome.

3. The detachable essential oil ring 10 may be detached from or mounted on the source device 20 easily to change the appearance of the lamp, and also allows users to change different flavor by using different detachable essential oil ring 10 with different essential oils easily. Thus, the flavors of different essential oils will not be mixed or mutual polluted by using the same detachable essential oil ring 10. In addition, the detachable essential oil ring 10 may absorb excess essential oils after a long time use and become dirty, and the user may remove the detachable essential oil ring 10 from the source device 20 easily to clean and to change a new one.

4. Different essential oils can be used with different detachable essential oil rings 10 to produce different taste, for example, the user can use different colors or shapes to distinguish different essential oils.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A detachable essential oil ring for lamp, the lamp has a heated portion during lighting, the detachable essential oil ring is an open-loop ring to movably mount on the lamp and having a top side, a part of the top side of the detachable essential oil ring absorbing or containing essential oils and contacting to the heated portion of the lamp to vaporize the essential oils by the heat generated from the lamp.

2. The detachable essential oil ring as claimed in claim 1, wherein the detachable essential oil ring has
a body being made of ceramic materials and having a top side, a part of the top side of the body being made without glazes to absorb or contain the essential oils; and
a notch formed through the body to form the open-loop detachable essential oil ring.

3. The detachable essential oil ring as claimed in claim 2, wherein
the body is C-shaped and has a protruding segment protruding from the body;
the protruding segment has at least one sheet element protruding from a periphery of the body; and
the at least one sheet element has a shape being circular, petal-shaped, cherry petal-shaped, leaf-shaped or polygonal.

4. The detachable essential oil ring as claimed in claim 3, wherein the body has an abutting segment axially formed on and protruding from the top side of the body to increase an contact area between the body and the heated portion of the lamp, where the heated portion is corresponding to a radiating shade of the lamp.

5. The detachable essential oil ring as claimed in claim 4, wherein
the protruding segment is connected to and protrudes from the body by sticking, engaging or forming;
the body is made of pottery clay, clay materials, ceramic materials, gypsum, porous thermally conductive materials or carbon materials to absorb or contain the essential oils; and
the abutting segment has a side face abutting the radiating shade of the lamp with glazes or thermal conductive layers.

6. A lamp comprising:
a source device having
a heated portion;
a radiating shade;
a light source mounted in the radiating shade to emit light and generate heat on the heated portion; and
a detachable essential oil ring being an open-loop ring, detachably mounted on the radiating shade and having a top side, a part of the top side of the detachable essential oil ring absorbing or containing essential oils and contacting the heated portion to vaporize the essential oils by the heat generated from the light source.

7. The lamp as claimed in claim 6, wherein
the radiating shade is made of optimum thermal conductivity material and has an external surface; and
a heat-dissipating face being semicircular and formed on the external surface of the radiating shade; and
the part of the top side of the detachable essential oil ring detachably contacts the heat-dissipating face of the heated portion to transmit heat from the light source to the detachable essential oil ring.

8. The lamp as claimed in claim 7, wherein the detachable essential oil ring has
a body being made of ceramic materials and having a top side, a part of the top side of the body being made without glazes to absorb or contain the essential oils; and
a notch formed through the body to foam the open-loop detachable essential oil ring.

9. The lamp as claimed in claim 8, wherein
the body is C-shaped and has a protruding segment protruding from the body;
the protruding segment has at least one sheet element protruding from a periphery of the body; and
the at least one sheet element has a shape being circular, petal-shaped, cherry petal-shaped, leaf-shaped or polygonal.

10. The lamp as claimed in claim 9, wherein
the body has an abutting segment axially formed on and protruding from the top side of the body to detachably contact the heat-dissipating face of the heated portion;
the protruding segment is connected to and protrudes from the body by sticking, engaging or forming;
the body is made of pottery clay, clay materials, ceramic materials, gypsum, porous thermally conductive materials or carbon materials to absorb or contain the essential oils;
the abutting segment has a side face contacting the heated portion of the lamp with glazes or thermal conductive layers; and
the light source is a light emitting diode.
